(19) United States
(12) Patent Application Publication
(10) Pub. No.: US 2021/0065946 A1
(43) Pub. Date: Mar. 4, 2021

(54) SUPERCONDUCTING MAGNET WITH THERMAL BATTERY

(71) Applicant: KONINKLIJKE PHILIPS N.V., EINDHOVEN (NL)

(72) Inventor: HONG HU, Watervliet, NY (US)

(21) Appl. No.: 16/960,110

(22) PCT Filed: Jan. 11, 2019
(86) PCT No.: PCT/EP2019/050619

§ 371 Date: Jul. 6, 2020

Related U.S. Application Data
(60) Provisional application No. 62/616,633, filed on Jan. 12, 2018.

(51) Int. Cl. H01F 6/04; H01F 6/06; A61B 5/055; G01R 33/3815; G01R 33/34

(52) U.S. Cl. CPC H01F 6/04; H01F 6/06; G01R 33/34023; G01R 33/3815; A61B 5/055

(57) ABSTRACT

A superconducting magnet includes a vacuum vessel (20), a liquid helium vessel (14) disposed in the vacuum vessel, and superconducting magnet windings (12) disposed in the liquid helium vessel. A thermal shield (22, 24) is spaced apart from and at least partly surrounds the liquid helium vessel. A thermal battery (30) is disposed in the vacuum vessel and is in thermally conductive contact with the thermal shield. The thermal battery may comprise a sealed container (32) in thermally conductive contact with the thermal shield and containing a working fluid such as nitrogen, and may contain a porous material (34). In operation, when active cooling of the magnet is turned off, the thermal battery slows the warming of the magnet by way of absorption of latent heat by the working fluid undergoing a solid-to-liquid or liquid-to-gas phase change.

### FIELD

The following relates to the superconducting magnet arts, magnetic resonance imaging (MRI) arts, thermal management arts, and related arts.

### BACKGROUND

In a typical superconducting magnet for a magnetic resonance imaging (MRI) system, the superconducting windings are cooled by liquid helium (LHe) in a LHe vessel disposed inside a vacuum vessel. A thermal shield of sheet material with high thermal conductivity is also disposed inside the vacuum vessel to surround the LHe vessel. The LHe vessel is spaced apart from the walls of the thermal shield, and in turn the thermal shield is spaced apart from the walls of the vacuum vessel, so that heat transfer from the ambient into the LHe vessel is suppressed as such heat must transfer by radiation inward from the vacuum vessel walls to the thermal shield and then by a further radiative transfer from the thermal shield to the LHe vessel. The vacuum of the vacuum vessel prevents conductive or convective heat transfer modes. After manufacture, the vacuum is drawn and the LHe vessel is filled with LHe. To maintain the LHe at cryogenic temperature (i.e. below 4K), a cold head is used to provide refrigeration to the LHe vessel. The first stage of the cold head penetrates through into the vacuum volume, and the first stage cold station is connected to the thermal shield by a high thermal conductance link that connects with a thermal bus attached to the thermal shield. The second stage of the cold head continues into the LHe vessel to cool the helium to below the temperature where helium liquefies (about 4.2K).

During shipment, the cold head is turned off and the magnet is shipped with the LHe charge loaded. With the cold head off, the vacuum jacket is relied upon to provide sufficient thermal insulation to maintain the LHe charge in its liquid state during shipping. In practice, the thermal shield typically increases fairly rapidly in temperature to around 100K with the magnet in transport mode with the cold head shut off. More generally, heat loss from the LHe vessel, mostly by radiation possibly with some conductive thermal losses through vessel support brackets or other conductive pathways, vaporizes a portion of the LHe charge. This limits the feasible transport distance, and/or requires operative hookup of the cold head during portions of the transport (which is not always possible if suitable electric power is unavailable), and/or requires adding additional LHe after the magnet arrives at the destination (which is costly and inconvenient).

In addition to transport, the cold head may be turned off for other reasons, such as to perform routine maintenance, magnet repair and/or testing, or due to inadvertent loss of electrical power. Also in such cases, excessive thermal leakage from the LHe charge can be problematic during prolonged periods in which the cold head is not operating. In these situations it is further possible that the superconducting magnet windings are conducting current in the superconducting state (i.e. the superconducting magnet is operating to provide a magnetic field). Here, loss of LHe can also lead to magnet quench, which can damage the magnet windings and requires restarting the magnet.

By way of illustration, in one superconducting magnet design a cold head is welded to the thermal shield and the LHe vessel. A disadvantage of this design is that the evaporation rate of the liquid helium is high when cold head is off. This adversely impacts the transportation time (and hence distance) for shipping the magnet from the factory to a customer site. Further, the high evaporation rate means that the magnet faces a heightened possibility of warming up sufficiently to quench the superconducting magnet when the cold head is off (intentionally or inadvertently, e.g. due to a power outage) during operation.

The following discloses certain improvements.

### SUMMARY

In some embodiments disclosed herein, a superconducting magnet comprises: a vacuum vessel; a liquid helium vessel disposed in the vacuum vessel and spaced apart from walls of the vacuum vessel; superconducting magnet windings disposed in the liquid helium vessel; a thermal shield disposed in the vacuum vessel and spaced apart from the walls of the vacuum vessel and spaced apart from and at least partly surrounding the liquid helium vessel; and a thermal battery disposed in the vacuum vessel and in thermally conductive contact with the thermal shield. The thermal battery may comprise a sealed container in thermally conductive contact with the thermal shield, and may further comprise a porous material disposed in the sealed container. The thermal battery may further comprise a working fluid filling the sealed container when in its gas phase, the working fluid having at least one (and optionally both of) of a gas/liquid phase transition temperature that is between 4K and 100K and a liquid/solid phase transition temperature that is between 4K and 100K. The working fluid is nitrogen in some embodiments. The sealed container may be welded to the thermal shield, and/or the thermal shield forms one wall of the sealed container. The superconducting magnet may further comprise a cold head including a motorized drive assembly, a first stage cold station thermally connected with the thermal shield or with the thermal battery, and a second stage cold station thermally connected with the liquid helium vessel.

In some embodiments disclosed herein, a magnetic resonance imaging (MRI) device comprises a superconducting magnet as set forth in the immediately preceding paragraph and arranged to generate a static Bo magnetic field in an examination region, and a set of magnetic field gradient coils for superimposing selected magnetic field gradients onto the static Bo magnetic field in the examination region.

In some embodiments disclosed herein, a superconducting magnet comprises: a vacuum vessel; a liquid helium vessel disposed in the vacuum vessel; superconducting coil windings disposed in the liquid helium vessel; a thermal shield disposed in the vacuum vessel and at least partially surrounding the liquid helium vessel; and a thermal battery disposed in the vacuum vessel and comprising nitrogen disposed in a sealed container that is in thermally conductive contact with the thermal shield.

In some embodiments disclosed herein, a method of operating a superconducting magnet is disclosed. The method comprises turning off active cooling of a liquid helium vessel containing magnet windings leading to warming of the superconducting magnet, and slowing the warming of the superconducting magnet using a thermal battery that is in thermally conductive contact with a thermal shield at least partly surrounding the liquid helium vessel of the superconducting magnet. The slowing may comprise slowing the warming of the superconducting magnet by absorption of latent heat by a working fluid of the thermal battery undergoing a solid-to-liquid and/or liquid-to-gas phase change due to the warming of the superconducting magnet. The method may further comprise, prior to turning off the active cooling: filling the thermal battery with a working fluid comprising nitrogen in a liquid state; and after the filling, turning on the active cooling whereby the liquid helium vessel is cooled to liquefy helium in the liquid helium vessel and the nitrogen in the liquid state is converted to nitrogen in a solid state.

One advantage resides in providing a superconducting magnet with reduced liquid helium (LHe) boil-off.

Another advantage resides in a superconducting magnet with reduced likelihood of quench during extended intervals over which the cold head is shut off.

Another advantage resides in providing a superconducting magnet that can be shipped over longer distances with a LHe charge.

Another advantage resides in providing a superconducting magnet that can have its cold head shut off for more extended time intervals to facilitate longer-distance shipping, extended maintenance, or so forth.

Another advantage resides in providing a superconducting magnet with reduced liquid helium evaporation during intervals over which the cold head is turned off or is non-operational.

Another advantage resides in providing a thermal shield for a superconducting magnet which provides more efficient thermal shielding to the LHe vessel, especially when active refrigeration is temporarily interrupted or shut off.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIGS. 1 and 2 diagrammatically illustrate side sectional and end sectional views, respectively, of a magnetic resonance imaging (MRI) system including a superconducting magnet with a thermal shield comprising a thermal battery as disclosed herein.

FIG. 3 diagrammatically illustrates an end sectional view of an alternative embodiment in which the thermal battery does not include a porous filler material.

FIG. 4 diagrammatically illustrates an end sectional view of an alternative embodiment in which the thermal battery is arranged as a plurality of longitudinal bars.

FIG. 5 diagrammatically illustrates a process flow for charging the superconducting magnet with liquid helium (LHe) and flow of events occurring in response to the cold head being turned off or losing electrical power.

### DETAILED DESCRIPTION

In embodiments disclosed herein, the thermal shield of a superconducting magnet comprises a thermal battery that uses the latent heat stored in nitrogen (or another working fluid such as hydrogen or dry air) to improve thermal performance. One or more phase changes of the working fluid of the thermal battery (e.g. solid-to-liquid phase transition, and/or liquid-to-gas phase transition) operates to absorb a portion of the heat load on the thermal shield during transportation mode (or other time when the cold head is not operating to provide active refrigeration). During the phase transition, the temperature of the working fluid remains at a fixed temperature. For example, solid nitrogen melts to liquid nitrogen (LN) at about 63K, and hence remains at 63K as the melting process absorbs latent heat. Similarly, LN vaporizes to nitrogen gas at about 77K, and hence remains at 77K as the vaporization process absorbs latent heat. During the solid-to-liquid phase transition, or the liquid-to-gas phase transition, the temperature of the working fluid remains constant, and hence the thermal shield remains at about that temperature. Therefore the heat load to liquid helium (LHe) vessel is reduced, and boil-off of LHe is expected to be reduced as well. The boil-off leads to the loss of the helium inside the LHe vessel of the superconducting magnet. No-sock magnets have relatively high boil-off rate, and the disclosed approach employs a thermal battery to reduce the boil-off rate. In the case of sealed magnet designs, the disclosed thermal battery increases the ride-through time by reducing the temperature increase on thermal shield when cold head is off.

In some illustrative embodiments, a thermal battery welded to the thermal shield comprises a sealed vessel containing a porous material (e.g. porous metal) and filled with nitrogen as the working fluid. When the cold head is not operating, the heat load on the thermal shield is absorbed by the metal porous material and nitrogen. As the nitrogen undergoes a solid-to-liquid or liquid-to-gas phase transition, it absorbs latent heat, maintaining the thermal shield at about the melting or evaporation temperature over the course of the phase transition and thereby retarding further temperature increase until the phase transition is complete. On the other hand, during normal magnet operation the thermal shield is typically cooled well below the 63K melting point of nitrogen (e.g. the thermal shield is expected to be around 35-40K during normal operation in some superconducting magnet designs) so the nitrogen is in the solid phase during normal operation of the magnet. Hence, the thermal battery does not impact normal operation.

During the manufacturing process, the nitrogen is injected into the thermal battery, e.g. as liquid nitrogen (LN). During magnet operation, the nitrogen is frozen solid. When active refrigeration by operation of the cold head stops (intentionally shut off or stops due to malfunction of the cold head or loss of electrical power to the cold head) the heat from the ambient (e.g., a room temperature of around 290-300K) will first melt the solid nitrogen and then evaporate the liquid nitrogen. From 300K to 80K range, the nitrogen will have two stages of phase change.

The disclosed approach employing a thermal shield comprising a thermal battery advantageously can be used alone or in combination with substantially any other thermal management configuration(s). The disclosed approach can also be used with any superconducting magnet design that utilizes a thermal shield. As the thermal shield is disposed in a vacuum vessel, it should be leak-tight to prevent leakage of nitrogen into the vacuum vessel which could compromise the vacuum and introduce detrimental conductive or convective heat transfer.

In one calculated design, a 600 mm wide, 10 mm thick thermal battery built on the thermal shield of one commercially available MRI superconducting magnet should take around 13 days to heat up the thermal shield to 77.35K in transportation mode. The boil-off of the helium is significantly reduced. For short distance transportation, the magnet with the disclosed improvement is expected to maintain zero boil-off (ZBO) even with cold head off.

In normal operation mode, the thermal battery reduce the temperature gradient on the thermal shield. Therefore, the thermal margin is improved.

In one contemplated manufacturing approach, the leak tight container of the thermal battery is welded to the 40K thermal shield. Liquid nitrogen is pumped into the thermal battery until it is full through the nitrogen refill and vent ports. The cold head is then turned on to actively refrigerate the thermal shield and thermal battery. The nitrogen inside will be cooled down to solid. The cooling energy is given off as latent heat during the liquid-to-solid phase transition. The thermal battery can be recharged in the field by refilling liquid nitrogen (if it has been lost through coupling leaks, or intentional venting for maintenance, et cetera) and turning on the cold head.

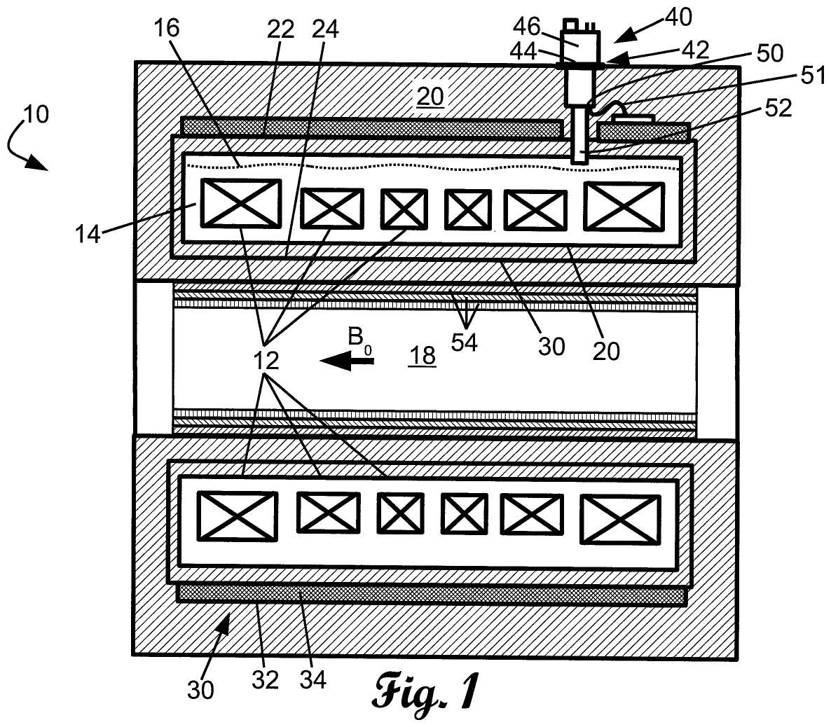

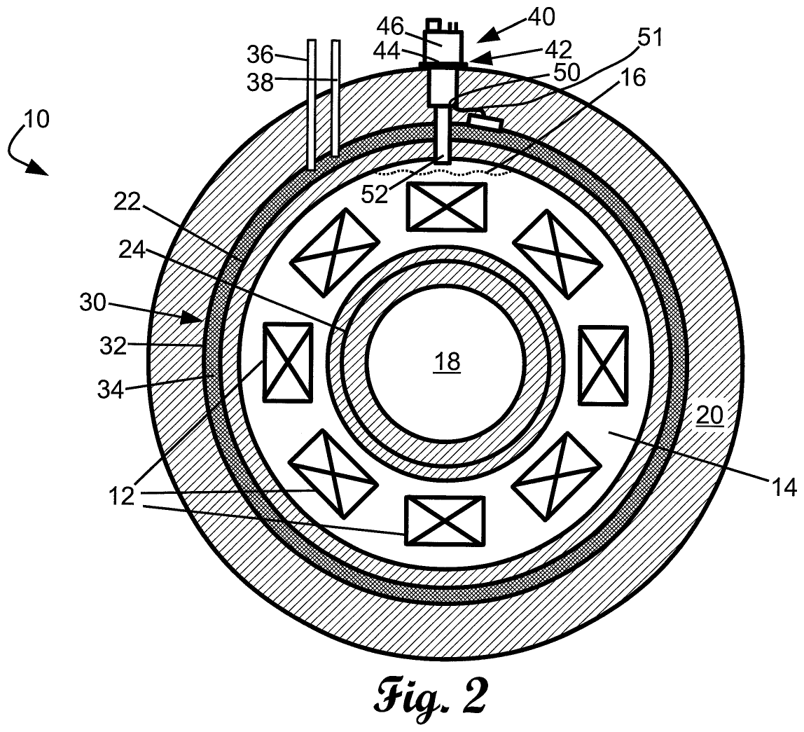

With reference now to FIGS. 1 and 2, a diagrammatic side sectional view (FIG. 1) and end sectional view (FIG. 2) is shown of an illustrative magnetic resonance imaging (MRI) device 10, which employs a superconducting magnet. The magnet includes superconducting coil windings 12 (for example, niobium-titanium or niobium-tin superconducting wires or filaments in a copper or copper alloy matrix, although other superconducting coil winding types are contemplated). The wire or tape itself may be made of tiny filaments (about 20 micrometers thick) of superconductor in a copper matrix) disposed in a liquid helium (LHe) vessel 14 which is mostly filled with LHe; however, there is a gaseous helium (gas He) overpressure present above the LHe level 16. The illustrative MRI device 10 employs a horizontal-bore magnet in which the superconducting magnet is generally cylindrical in shape and surrounds (i.e. defines) an examination region 18 in the form of a horizontal bore 18; however, other magnet geometries with otherwise shaped examination regions are also contemplated. To avoid conductive or convective heat transfer from ambient air to the LHe vessel 14, it is disposed inside a vacuum vessel 20. The vacuum volume contained by the vacuum vessel 20 is diagrammatically indicated in FIG. 1 by hatching.

To further thermally shield the LHe vessel 14, it is partly or entirely surrounded by a thermal shield 22, 24 which is also disposed inside the vacuum vessel 20. The thermal shield 22, 24 is disposed in the vacuum vessel 20 and spaced apart from the walls of the vacuum vessel 20, and the thermal shield 22, 24 is spaced apart from and at least partly surrounds the liquid helium vessel 14. The illustrative thermal shield 22, 24 includes an outer thermal shield wall 22 and an inner thermal shield wall 24 to provide thermal shielding for both the outer and inner circumferential walls of the cylindrical LHe vessel 14 of the illustrative horizontal-bore magnet. The thermal shield 22, 24 is preferably made of a sturdy thermally conductive material such as aluminum alloy sheet metal (or copper alloy sheet metal or some other high thermal conductivity sheet metal), and mostly or entirely surrounds the LHe vessel 14. The thermal shield 22, 24 is spaced apart from the walls of the vacuum vessel 20 to avoid thermal conduction from the thermal shield 22, 24 into the LHe vessel 14. (The thermal shield 22, 24 and the LHe vessel 14 may be structurally supported inside the vacuum vessel 20 by struts, brackets, or the like, not shown, which are designed to minimize thermal conduction by being made thin and/or made of material of low thermal conductivity). In some embodiments, the walls 22 and 24 of the thermal shield 22, 24 may comprise two or more sheets or layers (variant not shown) spaced apart from each other.

The illustrative thermal shield 22, 24 further includes, or is secured with, or comprises, a thermal battery 30 containing a working fluid that undergoes at least one phase transition (gas to liquid and/or liquid to solid) as the thermal shield decreases from ambient temperature to its operational temperature when the superconducting magnet is operational to carry superconducting magnet current in the magnet coils 12. Said another way, the thermal battery 30 comprises a working fluid having at least one of a gas/liquid phase transition temperature and a liquid/solid phase transition temperature that is between 4K (e.g. liquid helium temperature) and 100K. In some embodiments, the working fluid has both a gas/liquid phase transition temperature that is between 4K and 100K and a liquid/solid phase transition temperature that is between 4K and 100K. In the illustrative embodiments the working fluid is nitrogen, which undergoes a gas-to-liquid transition at about 77K and a liquid-to-solid transition at around 63K. Nitrogen has advantageous phase transition temperatures and is advantageously inexpensive. Other contemplated working fluids include hydrogen (gas/liquid transition temperature of about 20K and liquid/solid phase transition temperature of about 14K) or dry air (where the moisture should be sufficiently low to avoid generation of excessive water ice upon freezing; dry air has a gas/liquid transition temperature of about 79K and a liquid/solid phase transition temperature of about 58K). The illustrative thermal battery 30 includes a sealed container 32 containing the working fluid (e.g. nitrogen) that fills the sealed container 32 when in its gas phase. The container 32 should be hermetically sealed to prevent leakage of the working fluid into the vacuum volume of the vacuum vessel 20 supportive of conductive or convective heat transfer modes. The sealed container 32 is welded to (or otherwise in thermally conductive contact with) the thermal shield 22, 24. In the illustrative embodiment, the thermal shield wall 22 forms one wall 22 of the sealed container 32.

The illustrative thermal battery 30 further includes a porous material 34 disposed in the sealed container 32. For example, the porous material 34 can, for example, be porous aluminum or aluminum alloy, stainless steel, copper or copper alloy, alumina, or so forth. The porosity can be obtained in various ways, such as being in a granulated or pelleted or powdered form. The porous material 34 is optional, but is expected to improve the spatial uniformity of the phase transitions over the volume of the sealed container 32. The porosity of the porous material can be variously quantified, for example as a percentage of voids or open space, and the thermal capacity of the thermal battery is then the thermal capacity per unit volume of the working fluid times the volume of the sealed container times the voids percentage, assuming the working fluid can completely fill the voids or open space of the porous material. The porous material should preferably have mostly or completely interconnected voids or spaces between grains or pellets, or so forth, so that the working fluid (e.g. nitrogen) can readily fill the porosity.

To charge the sealed container 32 with nitrogen or another working fluid, a fill line 36 and vent line 38 is provided (shown in FIG. 2 but not FIG. 1). For example, in one contemplated charging sequence, liquid nitrogen is flowed into the sealed container 32 via the fill line 36 while displaced air and any vaporized nitrogen exits via the vent line 38. In a variant sequence, a vacuum is drawn via the vent line 38 before or while liquid nitrogen is flowed into the sealed container via the fill line 36. Other charging sequences are also contemplated.

With continuing reference to FIGS. 1 and 2, a cold head 40 executes a refrigeration cycle using a working fluid such as helium to provide active cooling of the LHe vessel 14, and also provides active cooling of the thermal shield 22, 24. The cold head 40 passes through the outer wall of the vacuum vessel 20 into the vacuum volume. A warm end 42 of the cold head 40 is welded to the outer wall of the vacuum vessel 20 by one or more welds 44. A motorized drive assembly 46 is connected to the warm end 42 of the cold head 40 (and may be viewed as part of the warm end 42), and includes a motor that drives a displacer (internal components not shown) to cause cyclic compression and expansion of the working fluid in accord with a refrigeration cycle. At least a portion of the motorized drive assembly 46 is outside of the vacuum jacket 20 and hence exposed to ambient air, and this includes connectors for attachment of one or more electrical power cables and one or more hoses for injecting the working fluid (cables and hoses not shown). The illustrative cold head 40 is a cylindrical cold head, although other geometries are contemplated. The illustrative cold head 40 is a two-stage design with a first stage cold station 50 and a second stage cold station 52. The first stage cold station 50 is connected by a thermal conductor 51 (e.g. copper braid, cable, or so forth) that is welded, brazed, or otherwise secured to the thermal shield 22, 24 directly or (as illustrated) to the attached thermal battery 30. (Note, if there is more than one thermal shield then the thermal battery is preferably secured with the thermal shield to which the first stage cold station is connected). The second stage cold station 52 penetrates into the liquid helium vessel 14 to thermally connect with and cool the liquid helium vessel. The cold head 40 is designed and operated to cool the second stage cold station 52 to below the temperature of liquid helium (about 4K), and the first stage cold station 50 to a higher temperature that is still low enough for the thermal shield 22, 24 to provide effective thermal shielding of the LHe vessel 14. In its operational (fully cooled) state, the first stage cold station 50 maintains the thermal shield 22, 24 and thermal battery 30 at a temperature low enough for the working fluid (e.g. nitrogen) of the thermal battery to be at least liquefied as LN, and in the illustrative embodiment to be solidified as solid nitrogen. More generally, in its operational state the first stage cold station 50 cools the working fluid so that, compared with room temperature (e.g. 290K), the working fluid has undergone at least one phase transition (e.g., if the working fluid is in the gas state at 290K then it is liquid or solid at the operational state; or, if the working fluid is in the liquid state at 290K then it is solid at the operational state). To provide vacuum-tight seals, the cold head 40 is typically welded to the outer wall of the vacuum vessel 20 and to the wall of the LHe vessel 14.

To operate the superconducting magnet, a LHe charge is loaded into the LHe vessel 14 via a suitable fill line (not shown). The fill line or another ingress path also provides for inserting electrical conductive leads or the like (not shown) for connecting with and electrically energizing the magnet windings 12. A static electric current flowing through these windings 12 generates a static Bo magnetic field, which is horizontal as indicated in FIG. 1 in the illustrative case of a horizontal bore magnet. After ramping the electric current in the magnet windings 12 up to a level chosen to provide the desired |B₀| magnetic field strength, the contacts can be withdrawn and the zero electrical resistance of the superconducting magnet windings 12 thereafter ensures the electric current continues to flow in a persistent manner. From this point forward, the LHe charge in the LHe vessel 14 should be maintained; otherwise, the superconducting windings 12 may warm to a temperature above the superconducting critical temperature for the magnet windings 12, resulting in a quench of the magnet. (To provide controlled shut-down in the event the LHe charge must be removed, the leads are preferably re-inserted and the magnet current ramped down to zero prior to removal of the LHe charge). The MRI device optionally includes various other components known in the art, such as a set of magnetic field gradient coils 54 (shown only in FIG. 1) for superimposing selected magnetic field gradients onto the Bo magnetic field in the examination region 18 in the x-, y-, and/or z-directions, a whole-body radio frequency (RF) coil (not shown) for exciting and/or detecting magnetic resonance signals, a patient couch (not shown) for loading a medical patient or other imaging subject into the bore 18 of the MRI device 10 for imaging, and/or so forth.

The cold head 40 chills the LHe vessel 14 when the cold head is operational. However, the cold head 40 is occasionally turned off. This may be done intentionally to prepare for maintenance, shipping of the magnet, or so forth, or may occur unintentionally due to some malfunction. Any time the cold head is turned off for any extended period of time, the loss of active refrigeration can result in heat from the ambient air at room temperature (typically at 290-300K or so) to radiate through the vacuum to warm the (no longer actively cooled) thermal shield 22, 24; and, as the thermal shield warms up, heat radiates from the thermal shield 22, 24 to the (no longer actively cooled) LHe vessel 14, causing boil-off of the LHe and, eventually, quenching of the magnet windings 12 if they are carrying a superconducting electrical current.

The thermal battery 30 advantageously slows the warming of the superconducting magnet by absorption of latent heat by the working fluid (e.g. nitrogen) of the thermal battery 30 undergoing a solid to liquid phase change due to the warming of the superconducting magnet, and further slows the warming of the superconducting magnet by absorption of latent heat by the working fluid (e.g. nitrogen) of the thermal battery 30 subsequently undergoing a liquid to gas phase change due to the warming of the superconducting magnet.

The illustrative thermal battery 30 of FIGS. 1 and 2 can have numerous variants while retaining the foregoing operation of slowing the warming of the superconducting magnet by absorption of latent heat by the working fluid (e.g. nitrogen) during solid-to-liquid and/or liquid-to-gas phase change(s).

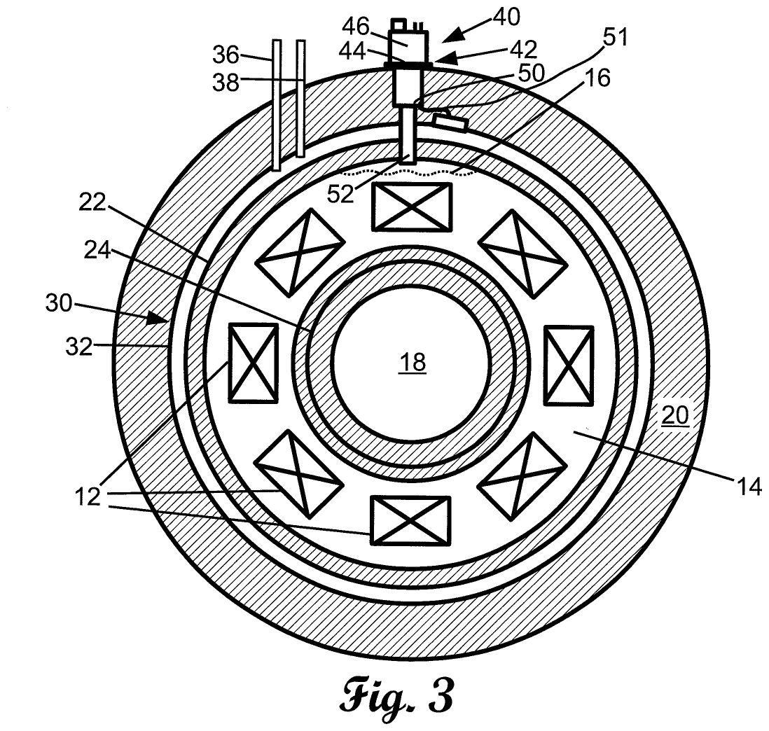

With reference to FIG. 3, an alternative embodiment is illustrated. This embodiment is identical with that of FIGS. 1 and 2, except that the porous material 34 is omitted. Thus, in the embodiment of FIG. 3, the sealed container 32 contains only the working fluid (e.g. nitrogen) but not the porous material 34.

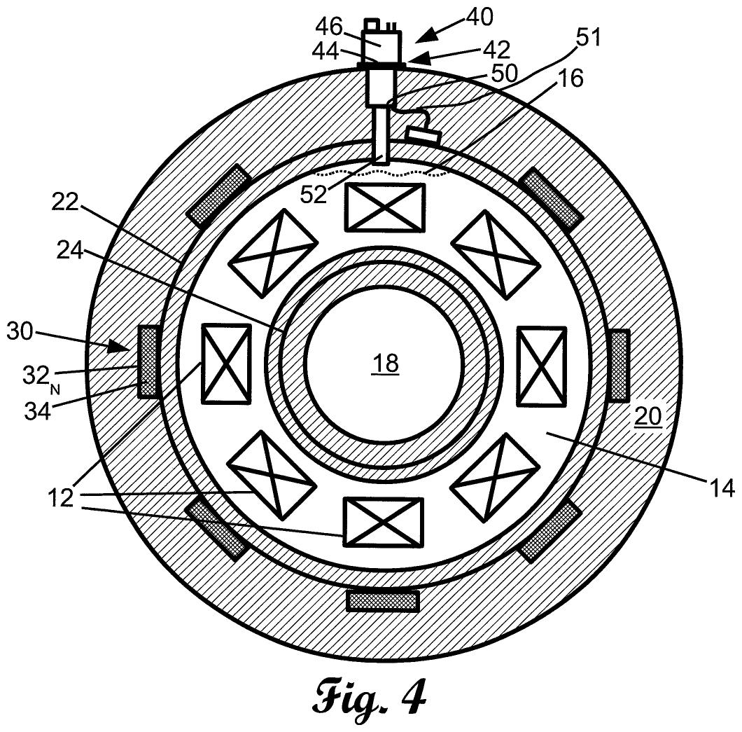

With reference to FIG. 4, another alternative embodiment is illustrated. This embodiment is identical with that of FIGS. 1 and 2 (and includes the porous material 34), but the sealed container 32 of the embodiment of FIGS. 1 and 2 is replaced in the embodiment of FIG. 4 by a plurality of sealed container sections 32_{N} each in thermally conductive contact with the thermal shield (22, 24). The number of sections 32_{N} is seven in illustrative FIG. 4, but can be more or fewer than this. FIG. 4 does not illustrate the fill and vent lines 36, 38, but these may be variously arranged in the embodiment of FIG. 4. For example, in one approach each sealed container section 32_{N} has its own fill and vent lines, and the sections are either filled in sequel or are filled simultaneously using suitable external manifold plumbing. In another approach, connecting pipes run between the sealed container sections 32_{N} by which they can be filled at the same time via a single set of fill/vent lines.

Each illustrative sealed container section 32_{N} comprises a rectangular bar shown in end view in FIG. 4, with the bar-shaped sealed container sections 32_{N} arranged in parallel and distributed around the circumference of the outer thermal shield wall 22. However, other geometric configurations are contemplated, such as the sealed container sections being a plurality of spaced-apart annular loops disposed around the outer thermal shield wall 22. As another contemplated variant (not shown) one or more sealed container sections could be welded to (or otherwise disposed on) the inner thermal shield wall 24.

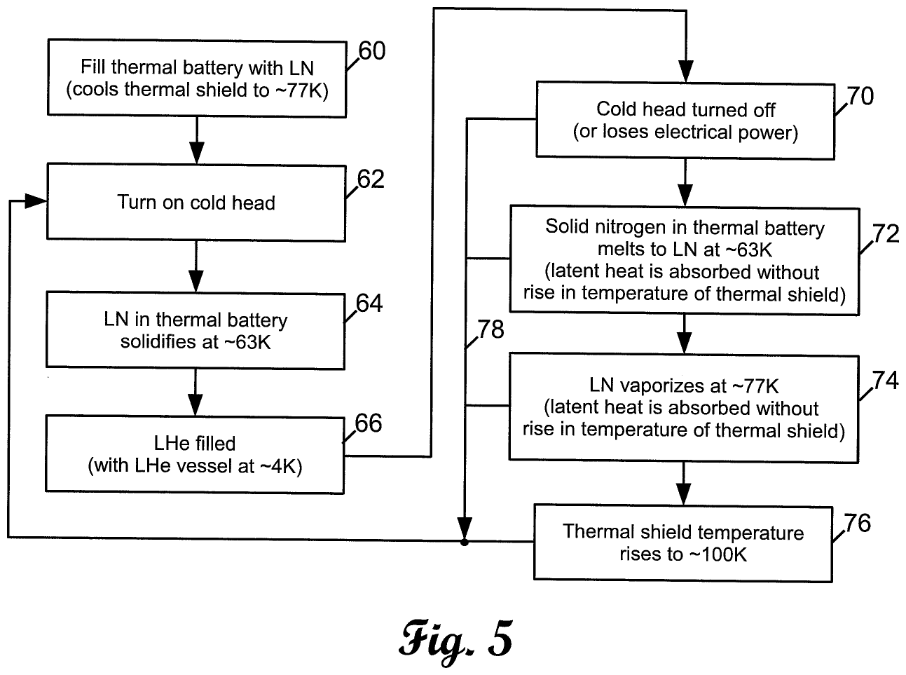

With reference to FIG. 5, an illustrative process is shown by which the superconducting magnet of FIGS. 1 and 2 (or, alternatively of FIG. 3, or of FIG. 4) may be operated. The left hand portion of FIG. 5 illustrates the cooldown phase. In an operation 60, the thermal battery 30 is filled with liquid nitrogen (LN). In other words, the nitrogen charge is already in liquid form. Accordingly, the thermal battery fill operation 60 operates to chill the battery 30 and the thermally connected thermal shield 22, 24 to the temperature of liquid nitrogen, i.e. about 77K. During the initial filling stage the liquid nitrogen will “blow off” as it vaporizes due to the sealed container 32 being initially at room temperature. The flowing liquid nitrogen cools the sealed container 32 in this way, and once the sealed container 32 reaches the temperature of liquid nitrogen it begins to fill with liquid nitrogen. After the thermal battery 30 is charged with nitrogen in the liquid phase, in an operation 62 the cold head 40 is turned on to initiate active cooling of the superconducting magnet. Due to operation of the cold head 40, the thermal shield 22, 24 is (further) cooled down by action of heat transfer to the first stage cold station 50 via the connecting thermal conductor 51, and concurrently the liquid helium vessel 20 is cooled by action of heat transfer to the second stage cold station 52 that is disposed in the helium or otherwise thermally connected with the liquid helium vessel 20.

As indicated by block 64, as the active cooling progresses the temperature of the thermal battery 30 eventually falls to the temperature (−63K) at which the liquid nitrogen undergoes a phase transition to solid nitrogen. At this point further cooling of the thermal shield 22, 24 will temporarily stop as the further cooling extracts latent heat from the liquid nitrogen to effect the phase transition to solid nitrogen. After the phase transition is complete, the sealed container 32 contains solid nitrogen (and, optionally, the porous material 34), at which point the active cooling via the first stage cold station 50 continues to lower the temperature of the thermal shield 22, 24. Concurrently, the liquid helium vessel 14 continues to cool by action of the second stage cold station 52, until the steady state is reached at which the helium in the helium vessel 14 is liquefied (except for an overpressure of gaseous helium) at a temperature of about 4K and the thermal shield 22, 24 reaches its steady state temperature, e.g. around 35-40K in some superconducting magnet designs. The steady state temperature of the thermal shield 22, 24 is not significantly affected by the thermal battery 30 at this point because the nitrogen is now solidified. Although not shown in FIG. 5, after reaching the steady state temperatures maintained by the active cooling, the superconducting coil windings 12 may be energized using known techniques to establish the persistent magnet current in the windings 12, thus providing the B₀ static magnetic field indicated in FIG. 1.

With continuing reference to FIG. 5, the right hand portion illustrates what happens when, as per operation 70, the cold head 40 is turned off preparatory to shipment of the magnet (or, alternatively preparatory to magnet maintenance, or alternatively, the operation 70 may represent an inadvertent loss of active cooling due to loss of electrical power to the cold head 40, or due to malfunction of the cold head 40, or so forth). The loss of active cooling at 70 leads to warming of the superconducting magnet. Initially, the liquid helium remains in liquid phase since the helium vessel 14 is in the vacuum provided by the vacuum vessel 20 (hence, not susceptible to significant heat ingress by thermal conduction or convection) and is thermally shielded by the thermal shield 22, 24. Thus, the helium vessel 14 initially remains at about the temperature of liquid helium, e.g. about 4K. However, the thermal shield 22, 24 and welded thermal battery 30 begin to warm up due to the radiative heat ingress from the surrounding walls of the vacuum vessel 20.

As indicated by block 72, this warming continues until the thermal battery 30 reaches the temperature at which the solid nitrogen transitions to the liquid phase, i.e. about 63K. At this point, the temperature rise is interrupted as the radiative heat is now instead absorbed as latent heat causing the phase transition from solid nitrogen to liquid nitrogen. After the solid nitrogen has transitioned to liquid nitrogen (so that the sealed container 32 now contains liquid nitrogen rather than solid nitrogen) the warming continues and the temperature of the thermal shield 22, 24 and welded thermal battery 30 begin to warm up again. This continues until, as indicated by block 74, the thermal battery 30 reaches the temperature at which the liquid nitrogen transitions to the gas phase, i.e. about 77K. At this point, the temperature rise is interrupted a second time as the radiative heat is now instead absorbed as latent heat causing the phase transition from liquid to gas. After the liquid nitrogen has transitioned to gas nitrogen (so that the sealed container 32 now contains gas nitrogen rather than liquid nitrogen) the warming continues and the temperature of the thermal shield 22, 24 and welded thermal battery 30 begin to warm up again.

Once the nitrogen in the thermal battery 30 has transitioned to gas, the thermal battery is no longer operative to slow the warming of the superconducting magnet. Eventually, the temperature difference between the helium vessel 14 and the warming thermal shield 22, 24 will lead to a liquid-to-gas transition of the helium, i.e. to helium boil-off and eventual quenching of the magnet windings 12 if they are carrying a persistent electrical current. In practice, however, it is expected that the cold head 40 will be turned back on before this happens, as indicated by flow lines 78 returning to the operation 62 at which the cold head is turned on. It will be appreciated that if the cold head is turned back on after the solid nitrogen has transitioned to liquid (block 72) but before the liquid nitrogen has transitioned to gas (block 74) then this liquid-to-gas transition will not be reached.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A superconducting magnet comprising:
a vacuum vessel;
a liquid helium vessel disposed in the vacuum vessel and spaced apart from walls of the vacuum vessel;
superconducting magnet windings in the liquid helium vessel;
a thermal shield disposed in the vacuum vessel and spaced apart from the walls of the vacuum vessel and spaced apart from and at least partly surrounding the liquid helium vessel; and
a thermal battery disposed in the vacuum vessel and in thermally conductive contact with the thermal shield.

2. The superconducting magnet of claim 1 wherein the thermal battery comprises a sealed container in thermally conductive contact with the thermal shield.

3. The superconducting magnet of claim 2 wherein the thermal battery further comprises a porous material disposed in the sealed container.

4. The superconducting magnet of claim 3 wherein the porous material comprises granulated, pelleted or powdered aluminum, aluminum alloy, stainless steel, copper, or copper alloy material.

5. The superconducting magnet of claim 2 wherein the thermal battery further comprises a working fluid filling the sealed container when in its gas phase, the working fluid having at least one of a gas/liquid phase transition temperature that is between 4K and 100K and a liquid/solid phase transition temperature that is between 4K and 100K.

6. The superconducting magnet of claim 2 wherein the thermal battery further comprises a working fluid filling the sealed container when in its gas phase, the working fluid having both a gas/liquid phase transition temperature that is between 4K and 100K and a liquid/solid phase transition temperature that is between 4K and 100K.

7. The superconducting magnet of claim 2 wherein the thermal battery further comprises nitrogen working fluid filling the sealed container when in its gas phase.

8. The superconducting magnet of claim 2 wherein at least one of:
the sealed container is welded to the thermal shield; or
the thermal shield forms one wall of the sealed container.

9. The superconducting magnet of claim 2 wherein the sealed container comprises a plurality of sealed container sections (32_{N}) each in thermally conductive contact with the thermal shield.

10. The superconducting magnet of claim 1 further comprising:
a cold head including a motorized drive assembly, a first stage cold station thermally connected with the thermal shield or with the thermal battery, and a second stage cold station thermally connected with the liquid helium vessel.

11. A magnetic resonance imaging (MRI) device comprising:
a superconducting magnet as set forth in claim 1 arranged to generate a static Bo magnetic field in an examination region; and
a set of magnetic field gradient coils for superimposing selected magnetic field gradients onto the static Bo magnetic field in the examination region.

12. A superconducting magnet comprising:
a vacuum vessel;
a liquid helium vessel disposed in the vacuum vessel;
superconducting coil windings disposed in the liquid helium vessel;
a thermal shield disposed in the vacuum vessel and at least partially surrounding the liquid helium vessel; and
a thermal battery disposed in the vacuum vessel and comprising nitrogen disposed in a sealed container that is in thermally conductive contact with the thermal shield.

13. The superconducting magnet of claim 12 wherein the thermal battery further comprises a porous material disposed in the sealed container.

14. The superconducting magnet of claim 13 wherein the porous material comprises granulated, pelleted or powdered aluminum, aluminum alloy, stainless steel, copper, or copper alloy material.

15. The superconducting magnet of claim 12 wherein the thermal shield comprises sheet metal and the sealed container is welded to the thermal shield.

16. The superconducting magnet of claim 12 wherein the thermal shield forms one wall of the sealed container.

17. The superconducting magnet of claim 12 wherein the sealed container comprises a plurality of sealed container sections (32_{N}) each in thermally conductive contact with the thermal shield.

18. The superconducting magnet of claim 12 further comprising:
a cold head including a motorized drive assembly, a first stage cold station thermally connected with the thermal shield or with the thermal battery, and a second stage cold station thermally connected with the liquid helium vessel.

19. A method of operating a superconducting magnet, the method comprising:
turning off active cooling of a liquid helium vessel containing magnet windings leading to warming of the superconducting magnet; and
slowing the warming of the superconducting magnet using a thermal battery that is in thermally conductive contact with a thermal shield at least partly surrounding the liquid helium vessel of the superconducting magnet.

20. The method of claim 19 wherein the slowing comprises at least one of:
slowing the warming of the superconducting magnet by absorption of latent heat by a working fluid of the thermal battery undergoing a solid-to-liquid phase change due to the warming of the superconducting magnet; and
slowing the warming of the superconducting magnet by absorption of latent heat by the working fluid of the thermal battery undergoing a liquid-to-gas phase change due to the warming of the superconducting magnet.

21. The method of any claim 19 wherein the slowing comprises:
slowing the warming of the superconducting magnet at least in part by absorption of latent heat by nitrogen of the thermal battery undergoing a solid-to-liquid phase change due to the warming of the superconducting magnet.

22. The method of any claim 21 wherein the slowing further comprises:
further slowing the warming of the superconducting magnet by absorption of latent heat by the nitrogen of the thermal battery undergoing a liquid-to-gas phase change subsequent to the solid-to-liquid phase change.

23. The method of claim 19 further comprising, prior to turning off the active cooling:
filling the thermal battery with a working fluid comprising nitrogen in a liquid state; and
after the filling, turning on the active cooling whereby the liquid helium vessel is cooled to liquefy helium in the liquid helium vessel and the nitrogen in the liquid state is converted to nitrogen in a solid state.
